(19) United States
(12) Patent Application Publication
(10) Pub. No.: US 2022/0151997 A1
(43) Pub. Date: May 19, 2022

(54) USE OF EPALRESTAT IN PREPARATION OF PANCREATIC CANCER DRUGS AND METHOD FOR VERIFYING INHIBITION EFFECT OF EPALRESTAT ON SECRETION OF EXOSOMES FROM PANCREATIC CANCER CELLS

(71) Applicant: Affiliated Hospital of Nantong University, Nantong (CN)

(72) Inventors: Mingbing XIAO, Nantong (CN); Yifei JI, Nantong (CN); Zhifeng GU, Nantong (CN); Wei SHI, Nantong (CN); Jie JI, Nantong (CN); Yihui FAN, Nantong (CN)

(73) Assignee: Affiliated Hospital of Nantong University, Nantong (CN)

(21) Appl. No.: 17/435,832

(22) PCT Filed: Mar. 24, 2020
(86) PCT No.: PCT/CN2020/080793

§ 371 Date: Sep. 2, 2021

(30) Foreign Application Priority Data
Apr. 17, 2019 (CN) 201910310233.4

(51) Int. Cl. A61K 31/426; G01N 1/30; G01N 33/50; A61P 35/00

(52) U.S. Cl. CPC A61K 31/426; A61P 35/00; G01N 33/5011; G01N 1/30

(57) ABSTRACT

The invention provides use of epalrestat in preparation of pancreatic cancer drugs. The pancreatic cancer drugs are used for inhibiting secretion of exosomes from pancreatic cancer cells. The invention also provides a method for verifying the inhibition effect of epalrestat on secretion of exosomes from pancreatic cancer cells. The method includes steps of: extracting cell supernatant exosomes by using a low-temperature ultracentrifugation method; lysing the collected exosomes, then using a BCA kit to quantify the resulting exosomal protein, and using the measured amount of the protein to reflect the amount of the exosomes; using a transmission electron microscope to verify a double-layer lipid membrane wrapped cup-shaped structure of the exosomes; and detecting exosome protein concentration by protein polyacrylamide gel electrophoresis with Coomassie brilliant blue. The invention provides a novel use of epalrestat, namely the inhibition of exosome secretion. The epalrestat has great application potential in clinical tumor treatment.

### TECHNICAL FIELD

The invention belongs to the technical field of epalrestat application, and in particular relates to use of epalrestat in preparation of pancreatic cancer drugs, and a method for verifying the inhibition effect of epalrestat on secretion of exosomes from pancreatic cancer cells.

### BACKGROUND

A special tumor microenvironment plays a key role in the occurrence, development, metastasis and drug resistance of pancreatic cancer. Pancreatic cancer helps its progress by secreting exosomes to shape the microenvironments of it and other organs. Compared with normal cells and other tumors, pancreatic cancer cells secrete more exosomes, and high-malignant pancreatic cancer cells can transfer their cancerous characteristics to low-malignant cancer cells through the exosomes, which promotes their proliferation, migration and invasion to accelerate disease progression. The exosomes derived from pancreatic cancer can also be taken up by Kupffer cells in the liver, causing Kupffer cells to secrete TGFP, thereby promoting production of fibronectin by hepatic stellate cells, and creating a microenvironment suitable for tumor growth and thus promoting liver metastasis. Therefore, it is of great significance for the clinical treatment of pancreatic cancer to find effective means to inhibit exosome secretion.

At present, the noncompetitive inhibitor GW4869 for neutral SMase (sphingomyelinase) is basically accepted as an exosome inhibitor, which can inhibit exosome secretion. In addition, some studies have found that in prostate cancer cells, the farnesyltransferase inhibitor Manumycin A inhibits the production and secretion of the exosomes by inhibiting the Ras signaling pathway and hnRNP H1 expression. Although all of these drugs can inhibit the production and release of the exosomes, they have not been tested clinically, and the development of a new drug needs to bear high investment and unknown risks. Therefore, how to find a new use of an old drug has become a problem to be solved at present.

### SUMMARY OF THE INVENTION

The technical problem to be solved by the invention is to provide use of epalrestat in the preparation of pancreatic cancer drugs and a method for verifying the inhibition effect of epalrestat on secretion of exosomes from pancreatic cancer cells, which has solved the problems raised in the background.

In order to solve the aforementioned technical problem, the embodiments of the invention provide use of epalrestat in preparation of pancreatic cancer drugs.

Further, the pancreatic cancer drugs are used for inhibiting secretion of exosomes from pancreatic cancer cells.

The embodiments of the invention provide a pharmaceutical composition against pancreatic cancer, wherein the pharmaceutical composition contains epalrestat.

Further, the pharmaceutical composition is used for inhibiting secretion of exosomes from pancreatic cancer cells.

The embodiments of the invention further provide a method for verifying the inhibition effect of epalrestat on secretion of exosomes from pancreatic cancer cells, characterized by including the following processes: (1) establishing a pancreatic cancer cell group treated with epalrestat and a pancreatic cancer cell control group, and extracting cell supernatant exosomes by using a low-temperature ultracentrifugation method; (2) lysing the collected exosomes, then using a BCA kit to quantify the resulting exosomal protein, and using the measured amount of the protein to reflect the amount of the exosomes; (3) using a transmission electron microscope to verify a double-layer lipid membrane wrapped cup-shaped structure of the exosomes; and (4) detecting exosome protein concentration by protein polyacrylamide gel electrophoresis with Coomassie brilliant blue.

Further, the step (1) specifically includes the following processes: separately taking 80 ml of cell supernatant from the pancreatic cancer cell group treated with epalrestat and the pancreatic cancer cell control group under the standard of the same number of cells; centrifuging at 4° C. and at 300×g for 15 min, at 2,000×g for 30 min, and at 16,500×g for 30 min, and removing the precipitate; filtering the obtained supernatant through a 0.22 μm filter, conducting low-temperature ultracentrifugation at 150,000×g for 120 min, collecting the precipitate, resuspending the precipitate in 100 μl PBS, subpackaging, and storing at −80° C.

Further, the step (2) specifically includes the following processes: taking 10 μL of the collected exosomes and fixing the exosomes with 2.5% glutaraldehyde for 2 h; washing the exosomes with PBS and resuspending the exosomes in 100 μL PBS; taking and adding dropwise 20 μl of the solution onto a small copper sheet, and conducting negative staining with an aqueous solution of 3% phosphotungstic acid for 1 min; and observing by a transmission electron microscope.

Further, the step (3) specifically includes the following processes: diluting a protein standard solution to 0.5 mg/ml with PBS; formulating a BCA working solution according to the sample size, wherein the BCA working solution is prepared immediately before use; adding the 0.5 mg/ml protein standard solution into a 96-well plate in the order of 0, 1, 2, 4, 8, 12, 16 and 20 μL, and supplementing to 20 μL with PBS, wherein the concentrations of the diluted standards are 0, 0.025, 0.05, 0.1, 0.2, 0.3, 0.4, and 0.5 mg/mL respectively; adding 1 μL of the exosome protein sample into each well, and adding PBS to supplement to 20 μL; and adding 200 μL of the BCA working solution into each well, incubating at 37° C. for 20-30 min, determining the absorbance at 562 nm with a microplate reader, and calculating the protein concentration of the sample according to a standard curve and the volume of the sample as used.

Further, the step (4) specifically includes the following process: preparing a 12% SDS-PAGE gel; adding a 5× loading buffer into the exosomes, and boiling for 20 min; loading the sample onto the gel; washing the gel with distilled water for 10 min after electrophoresis is completed; adding a proper amount of a rapid staining solution of Coomassie brilliant blue, shaking on a shaker for 1 h, and staining until a clear target protein band is seen; discarding the staining solution, adding a proper amount of distilled water, and taking pictures to observe the results.

The aforementioned technical solution of the invention has the following beneficial effects: the invention provides a novel use of epalrestat, namely, inhibiting exosome secretion, and in view of the key role of exosome secretion in tumors, epalrestat has great application potential in clinical tumor treatment.

### BRIEF DESCRIPTION OF THE DRAWINGS

FIG. 1A is a graph showing the morphology and quantity of exosomes in a pancreatic cancer cell control group as observed under a transmission electron microscope;

FIG. 1B is a graph showing the morphology and quantity of exosomes in a pancreatic cancer cell group treated with epalrestat as observed under a transmission electron microscope;

FIG. 2 is a graph showing the exosome protein concentrations of the pancreatic cancer cell control group and the pancreatic cancer cell group treated with epalrestat, as detected by a BCA method;

FIG. 3 is a diagram showing the exosome protein concentration bands as detected by protein gel electrophoresis with Coomassie brilliant blue staining;

FIG. 4 is a gray scale statistical diagram of exosome protein concentration bands as detected by protein gel electrophoresis with Coomassie brilliant blue staining;

FIG. 5A is a photograph showing the subcutaneous tumor formation situation in nude mice of the control group;

FIG. 5B is a photograph showing the subcutaneous tumor formation situation in nude mice of the experimental group; and

FIG. 6 is a diagram showing the expression amount of CD63 protein in exosomes of peripheral serum of nude mice as detected by dot immunoblotting.

### DETAILED DESCRIPTION OF THE EMBODIMENTS

In order to make the technical problems to be solved, technical solutions and advantages of the invention more apparent, the invention will be described in detail below in connection with the accompanying drawings and specific examples.

In the description of the invention, it should be noted that the directional or positional relationships indicated by the terms “center”, “upper”, “lower”, “left”, “right”, “vertical”, “horizontal”, “inner”, “outer”, “front”, “rear” and the like are based on the directional or positional relationships shown in the accompanying drawings, and are only for convenience of description of the present invention and simplification of the description, rather than indicating or implying that the indicated device or element must have a specific direction or be constructed and operate in a specific orientation, and therefore, cannot be understood as a limitation to the present invention. In addition, the terms “first”, “second” and “third” are only used for descriptive purposes, and cannot be understood as indicating or implying relative importance.

In the description of the present invention, it should be noted that unless otherwise specified and limited, the terms “installation”, “connection” and “connecting” should be understood in a broad sense, for example, they can be in fixed connection, detachable connection, or integrated connection; or they can be in mechanical connection or electrical connection; or they can be in direct connection, indirect connection through an intermediate medium, or communication of the interiors of two elements. For those of ordinary skills in the art, the specific meanings of the aforementioned terms in the invention can be understood according to specific situations.

Provided is use of epalrestat in preparation of pancreatic cancer drugs.

Further, the pancreatic cancer drugs are used for inhibiting secretion of exosomes from pancreatic cancer cells.

The embodiments of the invention provide a pharmaceutical composition against pancreatic cancer, wherein the pharmaceutical composition contains epalrestat.

Further, the pharmaceutical composition is used for inhibiting secretion of exosomes from pancreatic cancer cells.

The embodiments of the invention further provide a method for verifying the inhibition effect of epalrestat on secretion of exosomes from pancreatic cancer cells, characterized by including the following processes: (1) establishing a pancreatic cancer cell group treated with epalrestat and a pancreatic cancer cell control group, and extracting cell supernatant exosomes by using a low-temperature ultracentrifugation method; (2) lysing the collected exosomes, then using a BCA kit to quantify the resulting exosomal protein, and using the measured amount of the protein to reflect the amount of the exosomes; (3) using a transmission electron microscope to verify a double-layer lipid membrane wrapped cup-shaped structure of the exosomes; and (4) detecting exosome protein concentration by protein polyacrylamide gel electrophoresis with Coomassie brilliant blue.

In the embodiments of the invention, a method for verifying the inhibition effect of epalrestat on secretion of exosomes from pancreatic cancer cells specifically includes the following steps: extracting cell supernatant exosomes by using a low-temperature ultracentrifugation method; the detailed method was as follows: 80 ml of cell supernatant was separately taken from the pancreatic cancer cell group treated with epalrestat and the pancreatic cancer cell control group under the standard of the same number of cells, and centrifuged at 4° C. and at 300×g for 15 min, at 2,000×g for 30 min, and at 16,500×g for 30 min, and the precipitate was removed; the obtained supernatant was filtered through a 0.22 μm filter, low-temperature ultracentrifugation was conducted at 150,000×g for 120 min, the precipitate was collected and resuspended in 100 μl PBS, subpackaged, and stored at −80° C.

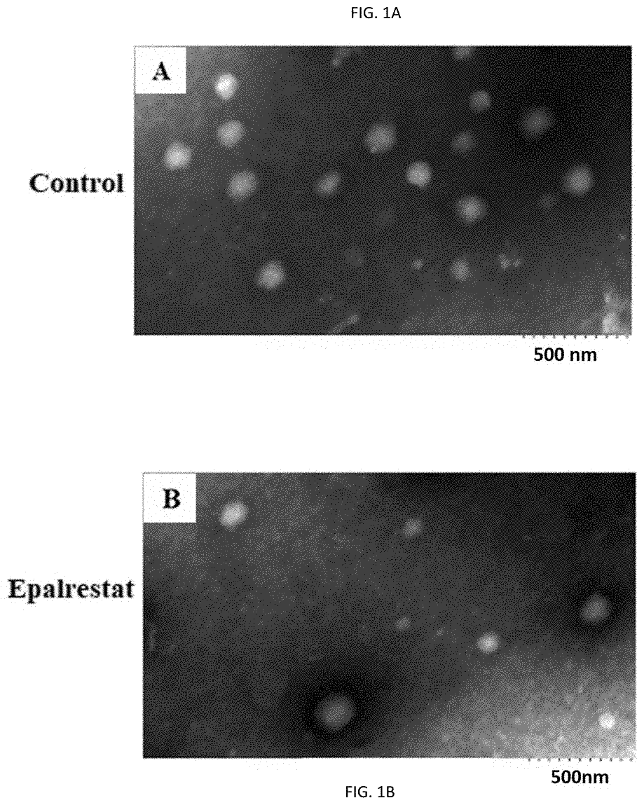

Using a transmission electron microscope to verify the double-layer lipid membrane wrapped cup-shaped structure of the exosomes, namely 10 μL of the collected exosomes were taken and fixed with 2.5% glutaraldehyde for 2 h; the exosomes were washed with PBS and resuspended in 100 μL PBS; 20 μl of the solution was taken and added dropwise onto a small copper sheet, and negative staining was conducted with an aqueous solution of 3% phosphotungstic acid for 1 min; and observation was conducted by a transmission electron microscope. The morphology and quantity of exosomes were observed by a transmission electron microscope, as shown in FIGS. 1A and 1B, wherein FIG. 1A is a graph showing the morphology and quantity of exosomes in a pancreatic cancer cell control group as observed under a transmission electron microscope; and FIG. 1B is a graph showing the morphology and quantity of exosomes in a pancreatic cancer cell group treated with epalrestat as observed under a transmission electron microscope.

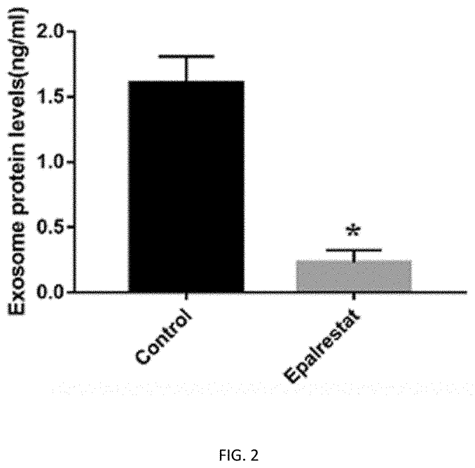

Lysing the collected exosomes, then using a BCA kit to quantify a protein, and using the measured amount of the protein to reflect the amount of the exosomes; the detailed method was as follows: the protein standard solution was diluted to 0.5 mg/mL with PBS; and a BCA working solution (a reagent A:a reagent B=50:1) was formulated according to the sample size, wherein the BCA working solution was prepared immediately before use. The 0.5 mg/ml protein standard solution was added into a 96-well plate in the order of 0, 1, 2, 4, 8, 12, 16 and 20 L, and supplemented to 20 L with PBS, wherein the concentrations of the diluted standards were 0, 0.025, 0.05, 0.1, 0.2, 0.3, 0.4, and 0.5 mg/mL respectively. 1 μL of the exosome protein sample was added into each well, and PBS was added to supplement to 20 μL. 200 μL of a BCA working solution was added into each well, and the mixture was incubated at 37° C. for 20-30 min. The absorbance at 562 nm was measured by a microplate reader. The protein concentration of the sample was calculated according to a standard curve and the volume of the sample as used. A BCA kit was used for quantifying the protein of the exosomes obtained by separation, as shown in FIG. 2, which was a statistical diagram of the exosome protein concentration in the pancreatic cancer cell control group as detected by a BCA method, wherein “Control” is exosome protein concentration data of the pancreatic cancer cell control group; and Epalrestat was the exosome protein concentration data of the pancreatic cancer cell group treated with epalrestat.

And detecting the exosome protein concentration by protein polyacrylamide gel electrophoresis with Coomassie brilliant blue; the detailed method was as follows: 12% SDS-PAGE gel was prepared. A 5× loading buffer was added into the exosomes, and boiled for 20 min, and then the sample was loaded onto the gel. The gel was washed with distilled water for 10 min after electrophoresis was completed. A proper amount of a rapid staining solution of

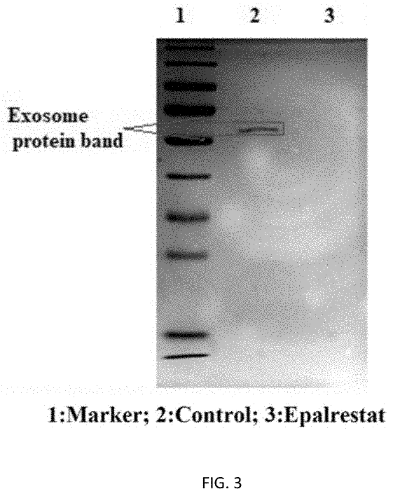

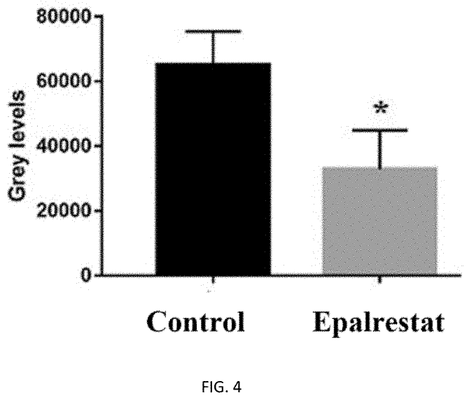

Coomassie brilliant blue was added, and shaking was performed on a shaker for 1 h. Staining was conducted until a clear target protein band was seen; the staining solution was discarded, a proper amount of distilled water was added, and pictures were taken to observe the results. The exosome protein concentration was detected by protein gel electrophoresis with Coomassie brilliant blue staining, as shown in FIGS. 3 and 4. FIG. 3 is a diagram showing the exosome protein concentration bands as detected by protein gel electrophoresis with Coomassie brilliant blue staining; and FIG. 4 is a gray scale statistical diagram of exosome protein concentration bands as detected by protein gel electrophoresis with Coomassie brilliant blue staining. The amount of exosomes is reflected by the measured protein amount. The aforementioned detection results showed: compared with the control group, the amount of the exosomes secreted from pancreatic cancer cells and the amount of proteins were decreased significantly after the aldo-keto reductase was inhibited. In FIG. 3, 1:Marker is a pre-staining protein marker, which was used as a standard for determining the molecular weight in SDS-PAGE (protein electrophoresis); 2:Control is the pancreatic cancer cell control group; and 3:Epalrestat is the pancreatic cancer cell group treated with epalrestat.

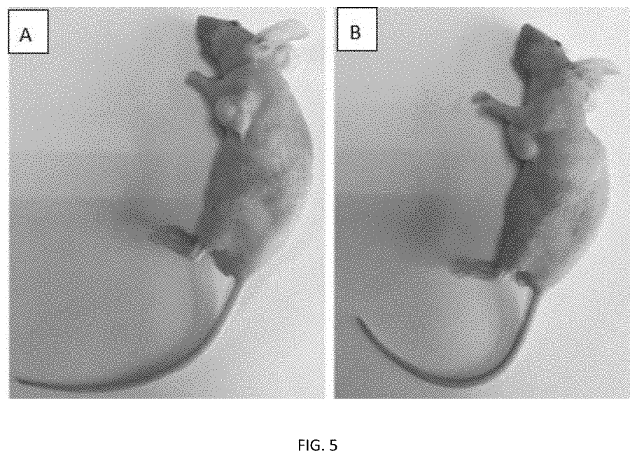

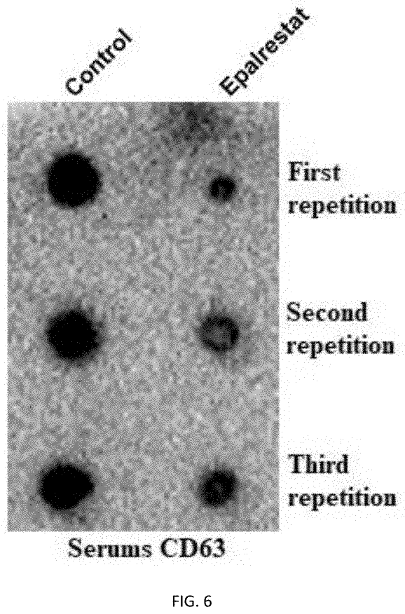

In order to verify that epalrestat could inhibit pancreatic cancer growth and exocrine secretion, a nude mouse experiment was carried out in the invention for verification, which specifically included the following steps: S1. Preparation of a nude mouse model: an untreated Capan-2 cell line was used for constructing a nude-mouse subcutaneous tumor formation model, wherein the model forming time was 20 days; S2. the nude mouse model was divided into an experimental group of nude mice and a control group of nude mice, with 10 nude mice in each group, and the nude mice of the experimental group were injected intraperitoneally with epalrestat at 50 mg/kg/d every day for three consecutive days; S3. 7 days later, the tumor volumes of the nude mice of the experimental group and the control group were observed and compared; and S4. The exosomes were extracted from the peripheral serum of the nude mice, and the expression amount of the exosome surface marker CD63 was detected by dot immunoblotting to reflect the amount of the exosomes. The photographs comparing the subcutaneous tumor formation situations in nude mice were as shown in FIGS. 5A and 5B. FIG. 5A is a photograph showing the subcutaneous tumor formation situation in nude mice of the control group; and FIG. 5B is a photograph showing the subcutaneous tumor formation situation in nude mice of the experimental group. It could be seen from the comparison between FIGS. 5A and 5B that the tumor volume of nude mice in the experimental group, i.e., the group intraperitoneally injected with epalrestat, became smaller. FIG. 6 is a diagram showing the expression amount of CD63 protein in exosomes of peripheral serum of nude mice as detected by dot immunoblotting, and the expression amount of CD63 was used for reflecting the amount of the exosomes, wherein “Control” is the expression of CD63 protein in the exosomes of peripheral serum in nude mice of the control group; Epalrestat is the expression of CD63 protein in the exosomes of peripheral serum of nude mice in the experimental group; and the first repetition, the second repetition and the third repetition are triplicates of an experiment. The detection results showed that: compared with the control group of nude mice, the amount of the exosomes secreted by the pancreatic cancer cells in the experimental group of nude mice (i.e., the group intraperitoneally injected with epalrestat) was decreased significantly.

In the present invention, the specific process of the dot immunoblotting method included the steps: (1) preparation: a negative pressure device was connected, the nitrocellulose membrane (NC membrane) was cut to a size equivalent to that of a 70-well plate and placed onto the 70-well plate, the negative pressure device was turned on, and the negative pressure value was set to 0.06 MPa; (2) sample addition: control wells and sample wells were set, and three duplicate wells were set for each well, 1 μl of control serum (mixed serum of several normal persons) was added into each control well, 1 μl of each sample to be tested was added into each sample well, and after the sample addition was completed, the NC membrane was air-dried for 30 min; (3) blocking: the NC membrane was placed in 5% skimmed milk powder (formulated with TBS-T), slowly shaken on a shaker, and blocked at room temperature for 1.5 h. The blocking buffer was discarded, and the NC membrane was rinsed with TBS-T for 2-3 times; (4) incubation of a primary antibody: the primary antibody was diluted with 5% BSA at a proportion of 1:500, the NC membrane was immersed into the diluted solution, shaken in a constant-temperature shaker, and incubated at 37° C. for 4 h. The NC membrane was placed into and washed with TB S-T for 3 times, each time for 5 min; (5) incubation of a secondary antibody: the secondary antibody was diluted with 1% skimmed milk powder at a proportion of 1:5000, and the NC membrane was immersed into the diluted solution, and incubated at room temperature for 1.5 h. The NC was placed into and washed with TBS-T for 3 times, each time for 15 min; and (6) development: the washed NC membrane was flatly spread at an appropriate position of a developing instrument, a developer was evenly added dropwise onto the membrane, and pictures of the membrane were taken by a gel imaging system and stored.

The reagents in the embodiments of the invention were prepared as follows:

Complete medium: 50 mL of fetal bovine serum (Gibco, USA) and 5 mL of a Penicillin Streptomycin solution (Gibco, USA) were added into 450 mL of a DMEM high-sugar medium (Hyclone), mixed uniformly, and then stored at 4° C.

Cell PBS (Sangon).

1× PBST buffer: 0.24 g of KH₂PO₄, 0.2 g of KCl, 1.44 g of Na₂HPO₄.12H₂O and 1 mL of Tween-20 were dissolved in deionized water, and the volume of the solution was made to a constant volume of 1000 mL, and stored at room temperature.

Primary antibody: Rabbit anti-mouse CD63 monoclonal antibody (Abcam, UK).

Secondary antibody: HRP-tagged goat anti-rabbit IgG (Jackson ImmunoResearch).

Epalrestat, an aldo-keto reductase inhibitor for preventing, improving and treating peripheral nerve disorders (numbness and pain) complicated by diabetes mellitus, has been widely used in clinic because of its low incidence of side effects, safety and effectiveness. In the invention, the novel use of epalrestat capable of significantly inhibiting secretion of exosomes from pancreatic cancer cells after blocking the polyol pathway is provided, and this characteristic enables epalrestat to probably have an important value in clinical tumor treatment, providing a new theoretical basis for clinical patients to find potential drug targets.

Epalrestat is an aldo-keto reductase inhibitor for treating diabetic complications clinically. Our results show that epalrestat has a novel use, namely, inhibiting exosome secretion, and in view of the key role of exosome secretion in tumors, epalrestat has great application potential in clinical tumor treatment.

The above descriptions are preferred implementations of the present invention. It should be noted that a person of ordinary skill in the art may further make several improvements and modifications without departing from the principle of the present invention, but such improvements and modifications shall also be deemed as falling within the protection scope of the present invention.

I claim:

1. A method for treating a patient with pancreatic cancer, wherein the method comprises the step of administering a drug containing epalrestat to the patient in need thereof.

2. The method according to claim 1, wherein the method comprises treating the patient by inhibiting secretion of exosomes from pancreatic cancer cells with the drug containing epalrestat.

3. A pharmaceutical composition against pancreatic cancer, wherein the pharmaceutical composition contains epalrestat.

4. The pharmaceutical composition against pancreatic cancer according to claim 3, wherein the pharmaceutical composition is used for inhibiting secretion of exosomes from pancreatic cancer cells.

5. A method for verifying the inhibition effect of epalrestat on secretion of exosomes from pancreatic cancer cells, wherein the method comprises the following processes: (1) establishing a pancreatic cancer cell group treated with epalrestat and a pancreatic cancer cell control group, and extracting cell supernatant exosomes by using a low-temperature ultracentrifugation method; (2) lysing the collected exosomes, then using a BCA kit to quantify the resulting exosomal protein, and using the measured amount of the protein to reflect the amount of the exosomes; (3) using a transmission electron microscope to verify a double-layer lipid membrane wrapped cup-shaped structure of the exosomes; and (4) detecting exosome protein concentration by protein polyacrylamide gel electrophoresis with Coomassie brilliant blue.

6. The method for verifying the inhibition effect of epalrestat on secretion of exosomes from pancreatic cancer cells according to claim 5, wherein the step (1) specifically comprises the following processes: separately taking 80 ml of cell supernatant from the pancreatic cancer cell group treated with epalrestat and the pancreatic cancer cell control group under the standard of the same number of cells; centrifuging at 4° C. and at 300×g for 15 min, at 2,000×g for 30 min, and at 16,500×g for 30 min, and removing the precipitate; filtering the obtained supernatant through a 0.22 μm filter, conducting low-temperature ultracentrifugation at 150,000×g for 120 min, collecting the precipitate, resuspending the precipitate in 100 μl PBS, subpackaging, and storing at −80° C.

7. The method for verifying the inhibition effect of epalrestat on secretion of exosomes from pancreatic cancer cells according to claim 5, wherein the step (2) specifically comprises the following processes: taking 10 μL of the collected exosomes and fixing the exosomes with 2.5% glutaraldehyde for 2 h; washing the exosomes with PBS and resuspending the exosomes in 100 μL PBS; taking and adding dropwise 20 μl of the solution onto a small copper sheet, and conducting negative staining with an aqueous solution of 3% phosphotungstic acid for 1 min; and observing by a transmission electron microscope.

8. The method for verifying the inhibition effect of epalrestat on secretion of exosomes from pancreatic cancer cells according to claim 5, wherein the step (3) specifically comprises the following processes: diluting a protein standard solution to 0.5 mg/ml with PBS; formulating a BCA working solution according to the sample size, wherein the BCA working solution is prepared immediately before use; adding the 0.5 mg/ml protein standard solution into a 96-well plate in the order of 0, 1, 2, 4, 8, 12, 16 and 20 μL, and supplementing to 20 μL with PBS, wherein the concentrations of the diluted standards are 0, 0.025, 0.05, 0.1, 0.2, 0.3, 0.4, and 0.5 mg/mL respectively; adding 1 μL of the exosome protein sample into each well, and adding PBS to supplement to 20 μL; and adding 200 μL of the BCA working solution into each well, incubating at 37° C. for 20-30 min, determining the absorbance at 562 nm with a microplate reader, and calculating the protein concentration of the sample according to a standard curve and the volume of the sample as used.

9. The method for verifying the inhibition effect of epalrestat on secretion of exosomes from pancreatic cancer cells according to claim 5, wherein the step (4) specifically comprises the following processes: preparing a 12% SDS-PAGE gel; adding a 5× loading buffer into the exosomes, and boiling for 20 min; loading the sample onto the gel; washing the gel with distilled water for 10 min after electrophoresis is completed; adding a proper amount of a rapid staining solution of Coomassie brilliant blue, shaking on a shaker for 1 h, and staining until a clear target protein band is seen; discarding the staining solution, adding a proper amount of distilled water, and taking pictures to observe the results.
